(19) United States
(12) Patent Application Publication
(10) Pub. No.: US 2008/0255430 A1
(43) Pub. Date: Oct. 16, 2008

(54) PORTABLE DEVICE WITH BIOMETRIC SENSOR ARRANGEMENT

(75) Inventors: Gustaf ALEXANDERSSON, Lund (SE); Krister THAM, Lund (SE)

Correspondence Address: HARRITY SNYDER, L.L.P., 11350 RANDOM HILLS ROAD, SUITE 600, FAIRFAX, VA, 22030, US

(73) Assignee: SONY ERICSSON MOBILE COMMUNICATIONS AB, Lund (SE)

(21) Appl. No.: 11/735,857

(22) Filed: Apr. 16, 2007

(51) Int. Cl. A61B 5/117

(52) U.S. Cl. 600/300

(57) ABSTRACT

The present invention is directed to a biometric sensor system 100 for measuring one or more physical or behavioral characteristic of a user, which system comprises: an information presenting unit 20 provided with a first sound presenting unit 22 and a second sound presenting unit 24 for operatively presenting sounds to a user, a portable device 10 provided with a sound producing unit 11, 40, 44, 46 for producing sounds to said sound presenting units 22, 24, and a biometric control unit 42 for controlling said measurement. The information presenting unit 20 comprises at least a first biometric sensor unit 32 and a second biometric sensor unit 32′ arranged to operatively enable a differential measurement of one or more physical or behavioral characteristic of a user, and the biometric control unit 42 is arranged to operatively control the biometric sensor units 32, 32′ so as to provide a differential measurement.

### TECHNICAL FIELD

The present invention relates to the field of portable devices and more particularly to portable devices comprising a biometric sensor arrangement for measuring one or more intrinsic physical or behavioral characteristic of a human. Particular aspects of the invention relate to a biometric sensor arrangement in connection with accessories to a portable device. Even more particular aspects of the invention relate to a biometric sensor arrangement in connection with accessories to a portable communication device.

### BACKGROUND OF THE INVENTION

It is generally known that biometric sensors can be used for measuring the physical or behavioral characteristic of a human and presenting the measured characteristic to a user. For instance, it is well known that a biometric sensor measuring the body temperature of a patient can be included in a handheld electronic device, of which a user puts the sensor part into the ear of a patient and reads a measured body temperature on an LCD display of the device. There is a vast spectrum of sensors that can be utilized for measuring the physical or behavioral characteristic of a human. Examples of physical or behavioral characteristic of a human that may be subjected to measurements may include but are not limited to body temperature, pulse (heart beats), brain activity (e.g. EEG, ElectroEncephaloGraphy), breathing frequency and transpiration etc.

However, it is often interesting to perform biometric measurements while a person is mobile, e.g. while walking, running or exercising in general. In such situations it is difficult to use a handheld device as described above. In addition, the handheld device is typically limited to a single area of use (e.g. temperature measurements), implying that another device is needed if another biometric characteristic is to be measured. Moreover, biometric measurements e.g. performed outdoors on a moving person are indeed more easily disturbed than ordinary measurements performed indoors under controlled conditions at a medical health centre. Measurements on a mobile or moving person may e.g. be disturbed by the person's movements and/or by the environment (sound, light, wind etc). Thus, the demand for interference resistance in a biometric sensor arrangement used for measurements on a mobile or moving person is considerably higher than in an ordinary biometric sensor arrangement. I addition, the monitoring of measured values is many times a difficult task when the person is mowing.

Thus, there seems to be a need for an improved biometric sensor system and a biometric sensor arrangement which enables a flexible biometric measurement on a mobile or moving person and which provides an improved measurement quality and preferably a feasible monitoring when the person is moving.

### SUMMARY OF THE INVENTION

The present invention is directed to solve the problem of providing an improved biometric sensor system and biometric sensor arrangement which enables a flexible biometric measurement on a mobile or moving person and which provides an improved measurement quality and preferably a feasible monitoring when the person is moving.

One object of the invention is thus to provide an improved biometric sensor system and biometric sensor arrangement which enables a flexible biometric measurement on a mobile or moving person and which provides an improved measurement quality and preferably a feasible monitoring when the person is moving.

This object is achieved according to a first aspect of the present invention which provides a biometric sensor system for measuring one or more physical or behavioral characteristic of a user, which system comprises: an information presenting unit provided with a first sound presenting unit and a second sound presenting unit for operatively presenting sounds to a user; a portable device provided with a sound producing unit for producing sounds to said sound presenting units, and a biometric control unit for controlling said measurement.

In particular, the information presenting unit comprises at least a first biometric sensor unit and a second biometric sensor unit arranged to operatively enable a differential measurement of one or more physical or behavioral characteristic of a user, and the biometric control unit is arranged to operatively control the biometric sensor units so as to provide a differential measurement.

It should be added that the sound producing unit may be a media player such as e.g. a radio receiver for streamed or broadcasted radio transmissions or an MP3 player for playing music pieces stored in the portable device or any other suitable player or similar arranged in the portable device. In addition, in case of a portable device in the form of a cell phone or similar the sound producing unit may additionally or alternatively be defined by the units that e.g. produce a ring signal to indicate an incoming call or the speech during a phone conversation etc.

A second aspect of the invention is directed to a portable biometric sensor system including the features of the first aspect, and wherein the biometric control unit is fully or at least partly arranged in the information presenting unit.

A third aspect of the invention is directed to a portable biometric sensor system including the features of the first aspect, and wherein the biometric control unit is arranged to operatively provide an average of the differential measurement from the first and second biometric sensors and a third biometric sensor arranged in the portable device.

A fourth aspect of the invention is directed to a portable biometric sensor system including the features of the first aspect, and further comprising a fastening arrangement for attaching at least a part of the information presenting unit to the body of the user.

A fifth aspect of the invention is directed to a portable biometric sensor system including the features of the first or fourth aspect, and wherein each of the sound presenting units is arranged to be operatively attached to an ear of a user.

A sixth aspect of the invention is directed to a portable biometric sensor system including the features of the first aspect, and wherein a first biometric sensor unit is attached to or included in the first sound presenting unit; and a second biometric sensor unit is attached to or included in the second sound presenting unit.

A seventh aspect of the invention is directed to a portable biometric sensor system including the features in any of the first, second, third, fourth, fifth or sixth aspect, and wherein the biometric sensor units are arranged to operatively sense the heart pulse, the EEG, EKG and/or body temperature of the user.

An eight aspect of the invention is directed to a portable biometric sensor system including the features of the seventh aspect, and wherein the biometric control unit is arranged to operatively present the sensed pulse in the form of audible pulse beats via the information presentation unit.

A ninth aspect of the invention is directed to a portable biometric sensor system including the features of the eight aspect, and wherein the biometric control unit and the sound producing unit are arranged to operatively present information to the user simultaneously via the information presentation unit.

A tenth aspect of the invention is directed to a portable biometric sensor system including the features in any of the first, second, third, fourth, fifth, sixth, seventh, eight or ninth aspect, and wherein the portable device is a cellular phone arranged be operatively connected to the information presenting unit by a wired or a wireless connection.

An eleventh aspect of the invention is directed to a portable biometric sensor system including the features in any of the first, second, third, fourth, fifth, sixth, seventh, eight, ninth or tenth aspect, and wherein the information presenting unit is an earphone arranged be operatively connected to the portable device by a wired or a wireless connection.

A twelfth aspect of the invention is directed to a portable biometric sensor system including the features in any of the first, second, third, fourth, fifth, sixth, seventh, eight, ninth, tenth or eleventh aspect, and further comprising another information presentation unit in the form of a display for presenting visible information to the user about the characteristic of the user measured by the biometric sensor units and the biometric control unit.

The object mentioned above is also achieved according to a thirteenth aspect of the present invention, which provides an information presenting unit provided with a first sound presenting unit and a second sound presenting unit for operatively presenting sounds to a user,

In particular, the information presenting unit comprises at least first biometric sensor unit and a second biometric sensor unit being arranged to operatively enable a differential measurement of one or more physical or behavioral characteristic of a user.

A fourteenth aspect of the invention is directed to an information presenting unit including the features in thirteenth aspect, and, wherein a biometric control unit is fully or at least partly arranged in the presenting unit for operatively control the biometric sensor units so as to provide a differential measurement.

A fifteenth aspect of the invention is directed to an information presenting unit including the features in the thirteenth aspect, and further comprising a fastening arrangement for attaching at least a part of the first information presenting unit to the body of the user.

A sixteenth aspect of the invention is directed to an information presenting unit including the features in thirteenth or fifteenth aspect, and, wherein each of the sound presenting units is arranged to be operatively attached to an ear of a user.

A seventeenth aspect of the invention is directed to an information presenting unit including the features in thirteenth aspect, and wherein
a first biometric sensor unit is attached to or included in the first sound presenting unit, and
a second biometric sensor unit is attached to or included in the second sound presenting unit.

An eighteenth aspect of the invention is directed to an information presenting unit including the features in any of the thirteenth, fourteenth, fifteenth, sixteenth, or seventeenth aspect, and wherein the biometric sensor units are arranged to operatively sense the heart pulse, the EEG, EKG and/or body temperature of the user.

A nineteenth aspect of the invention is directed to an information presenting unit including the features in any of the thirteenth, fourteenth, fifteenth, sixteenth, seventeenth or eighteenth aspect, and arranged to be operatively connected to a cellular phone by a wired or a wireless connection.

The object mentioned above is also achieved according to a twentieth aspect of the present invention, which provides a method for measuring one or more physical or behavioral characteristic of a user by a biometric sensor system comprising: an information presenting unit provided with a first sound presenting unit and a second sound presenting unit for operatively presenting sounds to a user; and portable device provided with a sound producing unit for producing sounds to said sound presenting units; and a biometric control unit for controlling said measurement,

The method is characterized by the steps of:
measuring one or more physical or behavioral characteristic of a user by said information presenting unit comprising at least a first biometric sensor unit and a second biometric sensor unit, and
controlling said measurement by a biometric control unit so as to provide a differential measurement.

A twenty-first aspect of the invention is directed to a method including the features of the twentieth aspect, and characterized by the steps of: controlling said measurement by a biometric control unit that is fully or at least partly arranged in the information presenting unit.

A twenty-second aspect of the invention is directed to a method including the features of the twentieth aspect, and characterized by the steps of: providing an average of the differential measurement from the first and second biometric sensors and a third sensor arranged in the portable device.

A twenty-third aspect of the invention is directed to a method including the features of the twentieth aspect, and characterized by the steps of: fastening at least a part of the first information presenting unit to the body of the user.

A twenty-fourth aspect of the invention is directed to a method including the features of the twentieth or twenty-third aspect, and characterized by the steps of: attaching the sound presenting units to the ears of a user.

A twenty-fifth aspect of the invention is directed to a method including the features of the twentieth aspect, and characterized by the steps of: performing said measuring by a first biometric sensor unit that is attached to or included in the first sound presenting unit, and a second biometric sensor unit that is attached to or included in the second sound presenting unit.

A twenty-sixth aspect of the invention is directed to a method including the features in any of the twentieth, twenty-first, twenty-second, twenty-third, twenty-fourth or twenty-fifth aspect, and characterized by the steps of: measuring the heart pulse, the EEG, EKG and/or body temperature of the user.

A twenty-seventh aspect of the invention is directed to a method including the features of the twenty-sixth aspect, and characterized by the steps of presenting the sensed pulse in the form of audible pulse beats via the information presentation unit.

A twenty-eight aspect of the invention is directed to a method including the features of the twenty-seventh aspect, and characterized by the steps of presenting information from the biometric control unit and the sound producing unit simultaneously to the user via the information presentation unit.

A twenty-ninth aspect of the invention is directed to a method including the features in any of the twentieth, twenty-first, twenty-second, twenty-third, twenty-fourth, twenty-fifth, twenty-sixth, twenty-seventh or twenty-eight aspect, and wherein the portable device is a cellular phone arranged be operatively connected to the information presenting unit by a wired or a wireless connection.

A thirteenth aspect of the invention is directed to a method including the features in any of the twentieth, twenty-first, twenty-second, twenty-third, twenty-fourth, twenty-fifth, twenty-sixth, twenty-seventh twenty-eight or twenty-ninth aspect, and wherein the information presenting unit is an earphone arranged be operatively connected to the portable device by a wired or a wireless connection.

A thirty-first aspect of the invention is directed to a computer program product stored on a computer usable medium, comprising readable program means for causing a portable device to execute, when said program means is loaded in the portable device comprising
an information presenting unit provided with a first sound presenting unit and a second sound presenting unit for operatively presenting sounds to a user,
a portable device provided with a sound producing unit for producing sounds to said sound presenting units, and
a biometric control unit for controlling said measurement,
   the steps of:
measuring one or more physical or behavioral characteristic of a user by said information presenting unit comprising at least a first biometric sensor unit and a second biometric sensor unit, and
controlling said measurement by a biometric control unit so as to provide a differential measurement.

A thirty-second aspect of the invention is directed to a computer program element having a program recorded thereon, where the program is to make a portable device to execute, when said program means is loaded in the portable device comprising
an information presenting unit provided with a first sound presenting unit and a second sound presenting unit for operatively presenting sounds to a user,
a portable device provided with a sound producing unit for producing sounds to said sound presenting units, and
a biometric control unit for controlling said measurement,
   the steps of:
measuring one or more physical or behavioral characteristic of a user by said information presenting unit comprising at least a first biometric sensor unit and a second biometric sensor unit, and
controlling said measurement by a biometric control unit so as to provide a differential measurement.

It should be emphasized that the term “comprises/comprising” when used in this specification is taken to specify the presence of stated features, integers, steps or components, but does not preclude the presence or addition of one or more other features, integers, steps, components or groups thereof.

It should also be emphasised that the methods defined by the appended claims may comprise further steps in addition to those mentioned and the steps may be performed in another sequence than the one given without departing from the present invention.

Further advantages of the present invention and embodiments thereof will appear from the following detailed description of the invention.

### BRIEF DESCRIPTION OF THE DRAWINGS

The present invention will now be described in more detail with reference to the enclosed drawings, in which:

FIG. 1 shows a schematic view of an exemplifying biometric sensor system 100 comprising a portable device in the form of a cell phone 10 and an earphone 20.

FIG. 2 shows the cell phone 10 and the sensor system 100 in FIG. 1 connected to a cellular network.

FIG. 3 shows a schematic illustration of the relevant parts of the cell phone 10 and the sensor system 100 in FIG. 1-2.

FIG. 4a shows a schematic front view of an earpiece 22 of the exemplifying earphone 20 in the sensor system 100 in FIG. 1-2 being connected to an ear 29 of a user.

FIG. 4b shows a schematic rear view of the earpiece 22 in FIG. 4a.

FIG. 4c shows a schematic side view of the earpiece 22 in FIG. 4a.

FIG. 5 is a schematic illustration of the light receiver 38 in the earpiece 22 and of a corresponding light receiver 38′ in the earpiece 24 of the earphone 20, schematically showing a differential measuring.

FIG. 6 is a schematic illustration of a data carrier in the form of an exemplifying CD ROM 70.

### DETAILED DESCRIPTION OF PREFERRED EMBODIMENTS

The present invention is directed towards a biometric sensor system and biometric sensor arrangement that enables a flexible biometric measuring on a mobile or moving person that provides a high interference resistance and preferably a feasible monitoring when the person is moving.

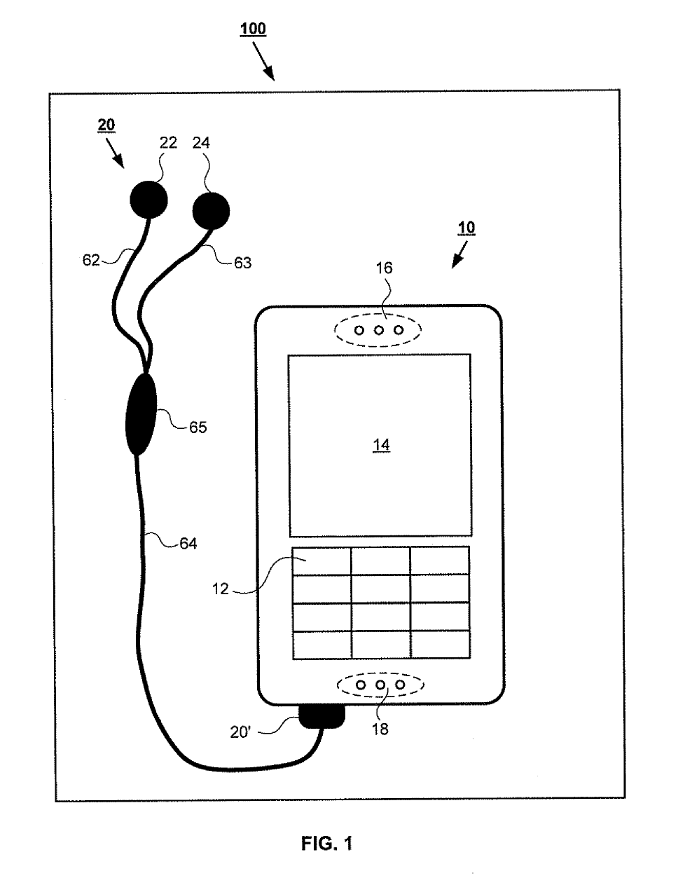

To this end a portable electronic device is shown in FIG. 1. In the preferred embodiment the device is a cellular phone 10 with a built-in antenna (not shown), a keypad 12 including a number of keys, a display 14, a speaker 16 and a microphone 18. The keypad 12 is used for entering information such as selection of functions and responding to prompts and the display 14 is used for displaying functions and prompts to a user of the phone 10. The speaker 16 is used for presenting sounds such as speech and/or music etc, whereas the microphone 18 is used for recording sounds such as speech and/or music etc.

The phone 10 is preferably provided with a system connector (not shown) into which a plug-in device 20 according to the invention can be connected. It is preferred that the system connector comprises a physical contact for connecting the plug-in device 20 to the phone 10. The system connector may be connected to one or several drivers and/or transducers arranged to operatively provide an interface between the plug-in device 20 and the information processing functions of the phone 10.

A portable biometric sensor system 100 according to an embodiment of the invention comprises the phone 10 and the plug-in device 20, while a biometric sensor arrangement according to an embodiment of the invention comprises the plug-in device 20 but not the phone 10.

As schematically illustrated in FIG. 1 the plug-in device is preferably an earphone 20, which at one end comprises a phone-connector 20′ for connecting the earphone 20 to the system connector of the phone 10, and which at the other end comprises two earpieces 22, 24 each being provided with a speaker arrangement for presenting sounds to a user of the earphone 20. As can be seen in FIG. 1 the phone-connector 20′ is preferably connected to a first cable 64, which splits into two cables 62 and 63 in turn being connected to the earpieces 22 and 24 respectively. More particularly, it is preferred that the first cable 64 and the two other cables 62 and 63 are connected a microphone and/or switch unit 65 preferably provided with electronic circuitry suitable for performing the functions required by the unit 65.

In addition, it is preferred that the embodiment of the present invention is based on an earphone 20 as shown in FIG. 1, which comprises at least two biometric sensor units for measuring one or more intrinsic physical or behavioral characteristic of a user, as will be described in more detail later.

Before we proceed it should be emphasised that a plug-in device such as the exemplifying earphone 20 shown in FIG. 1 does not have to be connected to the phone 10 by means of a wired connection. On the contrary, wireless plug-in devices such as e.g. Bluetooth devices or WLAN devices may be advantageously used in connection with the present invention.

The earphone 20 in FIG. 1 is a first information presenting unit comprising a first sound presenting unit in the form of an earpiece 22 and a second sound presenting unit in the form of an ear piece 24. In turn, the speaker 16 of the phone 10 is a second information presentation unit and the display 14 of the phone 10 a third information presentation unit. Thus, in this embodiment the first and second information presentation units 20, 16 are sound presentation units. It is preferred that at least one biometric sensor unit is arranged in each ear piece 22, 24 as will be described in more detail later. However, two or more biometric sensor units may be arranged elsewhere in connection with the earphone 20.

The earpieces 22, 24 of the earphone 20 are preferably adapted to be attached to the pinna (i.e. the visible part of the ear) of the user's ear. Some earphones may additionally be at least partly fitted in the opening of the ear canal or possibly even inside the ear canal of the user. Other manners for attaching the earphone 20 to the user's ear are clearly conceivable, e.g. by means of pads or similar that extends at least partly around the ear of the user, as is common for various headphones.

It should be emphasised that a cellular phone is just one example of a portable device in which the invention can be implemented. The invention can for instance also be used in a PDA (personal digital assistant), a palm top computer, a portable music playing device such as a CD player or an MP3 player or a cassette player. It can also be used in a portable radio. What is important is that it is possible to combine the biometric sensor arrangement (e.g. a plug-in device in the form of an earphone 20) with some type of information processing unit in a portable device.

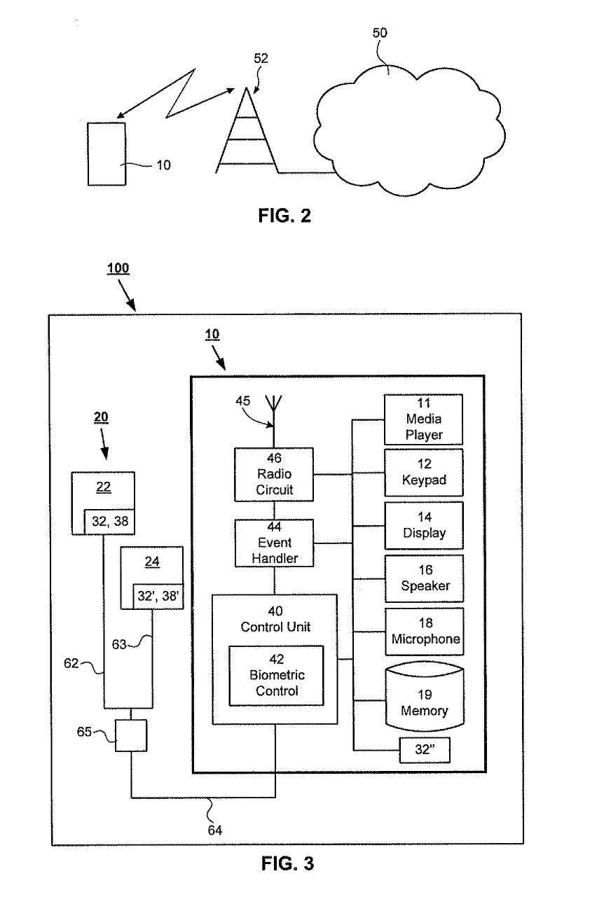

FIG. 2 shows the phone 10 connected to a cellular network 50 via a base station 52. The network 50 is preferably a 3G network such as e.g. a WCDMA network or similar, but it may also be a GPRS network or any other 2G, 2.5G or 2.75G network. Moreover, the network 50 does not have to be a cellular network but can be some other type of network, such as Internet, a corporate intranet, a LAN or a wireless LAN or similar.

FIG. 3 shows parts of the interior of the cell phone 10 being relevant for the present invention. As previously explained, it is preferred that the cell phone 10 is provided with a keypad 12, a display 14, a speaker 16 and a microphone 18. In addition, the cell phone 10 is preferably provided with a memory arrangement 19, e.g. for general storing purposes such as storing files and software programs to be used by the phone 10. The memory arrangement 19 may e.g. comprise one or several different memory units of any suitable kind that is commonly used in connection with portable devices. Moreover, the phone 10 I preferably comprising a media player 11—e.g. a radio receiver for streamed or broadcasted radio transmissions or an MP3 player or similar—for presenting sounds to the user via the speaker 16 or the earphone 20.

The cell phone 10 is also provided with an built-in antenna 45 connected to a radio circuit 46 for enabling radio communication with the network 50, as described above with reference to FIG. 2. The radio circuit 46 is in turn preferably connected to an event handling unit 44 arranged for handling such events as outgoing and incoming communication to and from external units via the network 50, e.g. calls and messages such as SMS (Short Message Service) and MMS (Multimedia Messaging Service) etc. In particular, data recorded or otherwise obtained by the phone 10—e.g. sounds recorded by the microphone 18 or images recorded by a possible phone-camera etc—may be transmitted as files or as a streamed transmission from the phone 10 to the network 50, e.g. by means of e.g. a connection to the Internet. The parts and functions of the phone 10 described above are well known to those skilled in the art and they are frequently implemented in modern cell phones. Hence, these parts and functions need no detailed description.

In connection with embodiments of the present invention it should be emphasised that data or similar being recorded or otherwise obtained by the phone 10 may in particular emanate from measurements performed by means of biometric sensor units preferably arranged in the earpieces 22, 24 of the earphone 20 connected to the system connector of the phone 10. The biometric sensor units etc will be described in more detail later.

Furthermore, as can be seen in FIG. 3, a control unit 40 is preferably arranged within the cell phone 10 for controlling and supervising the general operation of the phone 10. The control unit 40 may be implemented by means of hardware and/or software and it may comprise one or several hardware units and/or software modules, e.g. one or several processor units provided with or having access to the appropriate software and hardware required by the functions of the cell phone 10 as is well known by those skilled in the art. As can be seen in FIG. 3, it is preferred that the control unit 40 is connected to the media player 11, the keypad 12, the display 14, the speaker 16, the microphone 18 and the memory 19, the event handling unit 44, and the radio unit 46. This enables the control unit 40 to control and communicate with these units or similar to e.g. exchange information and instructions with the units. General control units like the control unit 40 described above are well known to those skilled in the art and they are frequently implemented in modern cell phones. Hence, there is no need for a detailed description of the parts and functions of the control unit 40.

Of particular interest in connection with the present invention is the biometric control unit 42 being added to the general control unit 40 described above. Being a part of the control unit 40 implies that the biometric control unit 42 may be implemented by means of hardware and/or software and it may comprise one or several hardware units and/or software modules, e.g. one or several processor units provided with or having access to the software and hardware appropriate for the functions required. The biometric control unit 42 is preferably arranged to operatively process the signals that is received from the biometric sensor units of the earphone 20.

In particular, the biometric control unit 42 is preferably arranged to operatively receive signals measured by biometric sensor units and to produce useful registrations of the measured physical and/or behavioral characteristic of the user, e.g. produce alphanumeric information on the display 14 indicative of said characteristic, or produce a data file to be stored in the memory 19 comprising measured samples of said characteristic, or produce data comprising measured samples of said characteristic to be streamed or similar from the phone 10 to the network 50, e.g. by means of a connection to the Internet or similar.

In addition, the control unit 40 may control the media player 11 or an incoming phone call so as to produce suitable sound signals representing various sounds enabling the speaker arrangements in the earpieces 22, 24 of an earphone 20 connected to the phone 10 to present an audible version of the sound to the user of the earphone 20, e.g. an audible version of a music piece digitally stored in the memory 19. Similarly, the cell phone 10 may receive music from a streamed radio program or similar via the network 50 as described above with reference to FIG. 2. In either case, the control unit 40 will forward suitable signals representing the sound from any received sound signals to the speaker arrangements in the earpieces 22, 24 of a connected earphone 20. Providing a suitable representation of various sound signals being received and/or produced by a cell phone or similar so that the speakers of an earphone or similar can reproduce an audible version to the user of the earphone or similar is well known per se and it needs not further description.

Before we proceed it should be emphasised that in addition to the parts and units shown in FIG. 3 there may be further parts and units or similar present in the cell phone 10. The parts and units shown in FIG. 3 may also be connected to more parts and units than those illustrated.

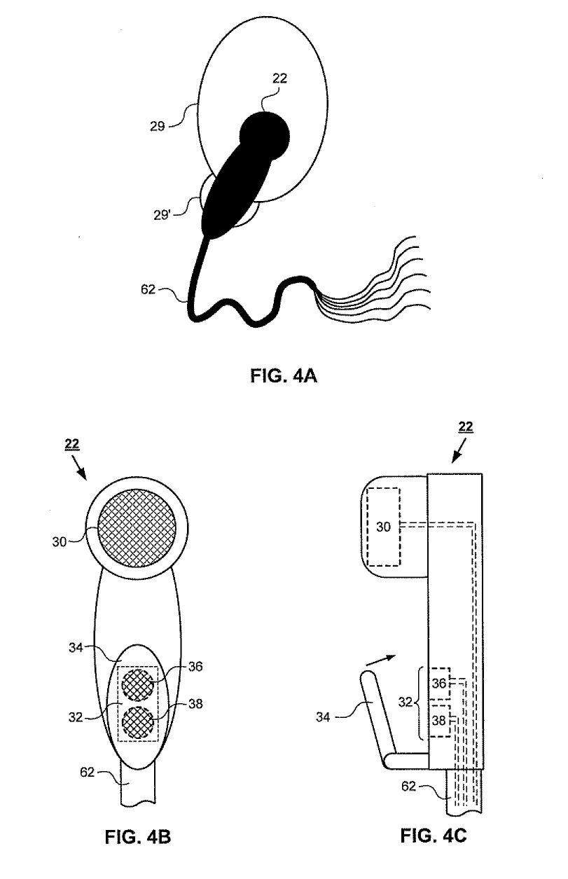

FIG. 4a is a schematic front view of the earpiece 22 of the earphone 20 being attached to an ear 29 of a user. The earpiece 22 is further connected to the phone 10 via the microphone and/or switch unit 65 and the cable 62 preferably including six wires, as will be explained in some detail later. The single earpiece 22 shown in FIG. 4a is preferably identical or at least nearly identical to the other earpiece 24 of the earphone 20 shown in FIG. 1, possibly with the exception for adaptations needed to meet differences between the human right and left ear. However, for the sake of simplicity the below description is mainly referring to the earpiece 22. It follows that the below description of earpiece 22 is applicable mutatis mutandis to the earpiece 24.

FIG. 4b is a schematic rear view of the earpiece 22. The earpiece 22 comprises a speaker arrangement 30 preferably adapted to be fitted in the pinna of the user's ear directly outside or at least near the entrance of the user's ear canal. Some earpieces may even be at least partly fitted in the opening of the ear canal and possibly even at least partly inside the ear canal. As already indicated above, other manners for attaching the earphone 20 to the user's ear are clearly conceivable, e.g. by means of pads or similar that extends at least partly around the pinna of the user ear as is common in connection with various headphones.

In addition to the speaker arrangement 30 discussed above the earpiece 22 comprises a biometric sensor unit. In FIG. 4b the biometric sensor unit has been exemplified by a pulseoximeter unit 32 arranged below the speaker arrangement 30. A pulsoximeter is essentially an optical device used for a non-invasive measurement of heart beat rate (pulse) and oxygen saturation etc in arterial human or animal blood. Pulseoximeters are e.g. described in the patent documents EP 0 781 527 (Weckström) and US 2005/0049468 (Carlsson), which are hereby incorporated by reference.

Furthermore, it is preferred that the earpiece 22 comprises a clamp-arrangement 34 or some other suitable fastening arrangement for closely attaching the pulseoximeter unit 32 against the ear 29 of the user and more preferably against the earlobe 29′ of the user's ear 29. The earlobe 29′ of the user's ear 29 is schematically illustrated in FIG. 4a. As can be seen in FIG. 4b the exemplifying clamp-arrangement 34 is preferably arranged in front of the pulseoximeter unit 32.

FIG. 4c is a schematic side view of the ear piece 22 showing the speaker arrangement 30 of the earpiece 22 provided above the pulseoximeter unit 32. As can be seen, the speaker arrangement 30 has two wires (indicated by dashed lines) for connecting to the information processing functions of the phone 10—e.g. the control unit 40—via the phone connector 20′ of the earphone 20 and the system connector of the phone 10 as described above with reference to FIG. 1. Similarly, the pulseoximeter unit 32 has four wires (indicated by dashed lines) for connecting to the biometric control unit 42 of the phone 10 via the phone connector 20′ of the earphone 20 and the system connector of the phone 10. It is preferred that the pulseoximeter unit 32 includes a light transmitter 36 and a light receiver 38, e.g. an IR (Infrared) transmitter and receiver. Two of the wires are intended for the light transmitter 36 and two wires for the light receiver 38. However, the number of wires can be varied. For example, there might be only three wires to the pulseoximeter unit 32. In the latter case the light transmitter 36 and light receiver 38 may share a common ground wire. However it should be emphasised that the ear piece 22 may be a wireless device or at least a part of a wireless device, e.g. a Bluetooth device.

As already indicated, optical pulsoximetry is used for non-invasive measurement of heart beat rate and oxygen saturation etc in the human body. In this connection it is preferred that the light source 36 is emitting light at two wavelengths, e.g. at a first wavelength of substantially 660 nm and at a second wavelength of substantially 890 nm. It is also preferred that the light receiver 38 detects the light transmitted through the earlobe, i.e. through the tissue portion of the person to be surveyed. To this end the light transmitted by the light transmitter 36 may be reflected back to the light receiver 38, e.g. reflected by the illuminated tissue and/or by the clamp-arrangement 34. Alternatively, it is possible to arrange the light transmitter 36 and the light receiver 38 opposite to each other. For example, the light transmitter 36 may be arranged as indicated in FIG. 4c, whereas the light receiver 38 may be arranged in the clamp-arrangement 34 or the other way around.

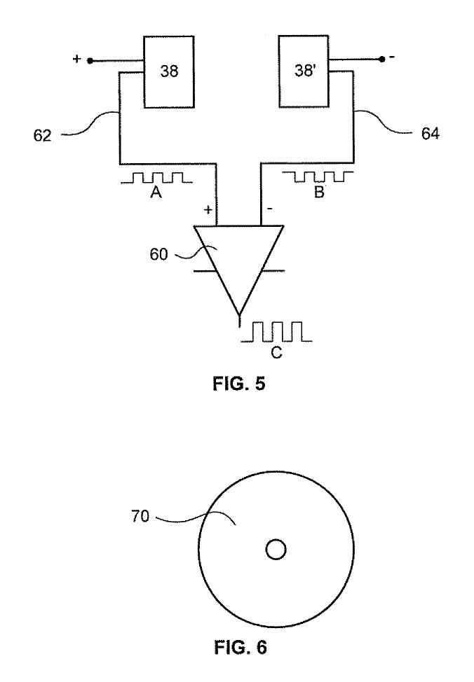

FIG. 5 is a schematic illustration of the light receiver 38 in the pulseoximeter unit 32 of the earpiece 22, and of a corresponding light receiver 38′ in a corresponding pulseoximeter unit 32′ in the earpiece 24 of the earphone 20 (see FIG. 3). In addition, a schematic differential amplifier 60 is shown, comprising a first input terminal marked with a plus sign and a second input terminal marked with a minus sign. The amplifier 60 is arranged to operatively amplify the difference between the two input terminals. The differential amplifier 60 may e.g. be an operational amplifier. The amplifier 60 or similar may be physically located in the earphone 20 and/or in the phone 10.

As can be seen in FIG. 5 the first light receiver 38 is connected by a first wire in the cable 62 to the first input terminal of the amplifier 60, whereas the second light receiver 38′ is connected by a second wire in the cable 64 to the second input terminal of the amplifier 60. Here, it is supposed that the signals from the two light receivers 38, 38′ are substantially the same. Both signals may e.g. comprise information about substantially the same pulse beat measured substantially simultaneously on a user of the earphone 20 and the phone 10 as described above. However, the signals from the two light receivers 38, 38′ are preferably opposite to each other, i.e. one signal is inverted compared to the other. This has been illustrated in FIG. 5 by a first schematic square signal A and a second schematic square signal B being 180° out of phase with respect to each other. Naturally, the real world signals may have other shapes. Since the first signal A and the second signal B are substantially opposite to each other the differential amplifier 60 will amplify the difference between the signals A, B and produce a signal C with substantially twice the amplitude, provided that the amplification is set to one. However, as the observant reader realises any common noise or similar disturbance equally induced in the wires of the two cables 62, 64 will arrive at the differential amplifier 60 substantially without any phase difference and the noise or similar will therefore be cancelled out by the differential amplifier 60, since there is substantially no difference between the two signal to be amplified.

From the above it follows that utilizing two light receivers 38, 38′—or two other suitable biometric sensors for that matter each being arranged in an earpiece 22, 24 of the earphone 20 for a differential measurement—provides an improved measurement quality, i.a. due to an improved interference resistance. One may say that utilizing two biometric sensor units each arranged in an earpiece of a plug-in unit for a differential measurement provides an improved measurement quality, i.a. due to an improved interference resistance.

Now the function of a preferred embodiment of the present invention will be explained in some detail. A user of the phone 10 and the earphone 20 will put each of the earpieces 22, 24 into an ear, as is well known in connection with common earphones. Next, the clamp-arrangement 34 of each earpiece 22, 24 or some other suitable fastening arrangement is arranged so as to closely attach the pulseoximeter unit 32 of each earpiece 22, 24 against an ear 29 of the user and more preferably against the earlobe 29′ of the user's ear 29. However, as an alternative or as a complement to the clamp-arrangement 34 the earpiece 22, 24 may be at least partly fitted in the opening of the ear canal or possibly even inside the ear canal of the user. The ear canal environment is very good for measurement, since it is quite humid and in addition provides a stable connection which does not move even if the user is moving quite a lot. Naturally, a humid environment may be particularly advantageous when measuring electrical signals such as e.g. EKG (ElectroCardioGram) or EEG (ElectroEncephaloGraphy) or similar. In addition, measuring body temperature in the ear canal is likewise advantageous du to its humid, heat conducting and substantially undisturbed environment.

Once attached the pulseoximeter units 32 of the earpieces 22, 24 are held in a measuring position close to the ear of the user or rather the earlobe of the user. Pulse metering by means of the pulseoximeter unit 32 can then be activated, for instance via selection of pulse metering in a suitable menu of the phone 10, i.e. by actuating ON/OFF buttons of the phone 10 or other similar buttons. In addition, a MP3 player or some other sound source comprised by the pone 10 may also be activated such that the user can use the phone 10 as a music playing device while moving, e.g. while exercising.

In operation the pulseoximeter unit 32 of each earpiece 22, 24 measure the pulse etc of the user by means of the light transmitter 36 and the light receiver 38 as is well known in connection with pulseoximeters. The measurement results are provided to the biometric control unit 42. The biometric control unit 42 may then determine the pulse etc of the user and present it to the user.

The pulse may e.g. be presented as a number of pulse beats via the speaker arrangement 30 of at least one of the earpieces 22, 24, possibly in the background of the music being played as e.g. a number of tones having lower pitch than the music being played. Alternatively or additionally, the pulse beats may be presented via the speaker 16 of the phone 10.

The biometric control unit 42 may additionally or alternatively present the pulse beats on the display 14 of the phone 10, e.g. as number of beats per minute or number of beats according to any other suitable time period. In this case the biometric control unit 42 has to calculate the value based on a number of measurements. The biometric control unit 42 may also measure the pulse during a certain longer period, for instance during a jogging session, and then present the variations later.

Various measurements performed by a biometric sensor unit—e.g. a pulseoximeter unit 32 as described above—and/or results based on such measurements may be stored in the phone 10 and/or be transmitted as files or as a streamed transmission from the phone 10 to the network 50, e.g. by means of e.g. a connection to the Internet.

Providing a plug-in device (e.g. an earphone 20) of a portable device (e.g. a cell phone 10) with a biometric sensor unit (e.g. a pulseoximeter unit 32) enables a flexible biometric measurement that can be used on a mobile or moving person. In addition, providing a biometric sensor in each earpiece 22, 24 or similar of an earphone 20 or similar provides a flexible biometric measurement on a mobile or moving person and a differential measurement which gives an improved measurement quality. An improved measurement quality is a particularly important factor in connection with measurements on mobile or moving persons, since the measurement may be disturbed by the person's movements and/or by the shifting in the environment (sound, light, wind etc) as the person moves.

In addition, selecting the ear for measuring the pulse in particular provides a much better place for measuring compared with for instance the fingertip as is commonly used for measuring pulse. The ear is a part of the human body, which gives very good results for pulse measurements. In addition, the ear canal in particular environment is very good for measurement, since it is quite humid and in addition provides a stable connection which does not move even if the user is moving. A humid environment may be particularly advantageous when measuring electrical signals such as e.g. EKG (ElectroCardioGram) or EEG (ElectroEncephaloGraphy) or similar. In addition, measuring body temperature in the ear canal is likewise advantageous du to its humid, heat conducting and substantially undisturbed environment. By selecting the ear it is furthermore possible to combine a biometric measurement with listening to media such as recorded music or a radio station. Yet another advantage is that a user only has to keep track of one device connected to his body instead of two different ones, which can be a problem during exercise.

The invention may be varied in many ways. For instance the plug-in device (e.g. the earphone 20) may fully or at least partly include the biometric control unit 42, e.g. integrated in the microphone and/or switch unit 65 shown in FIGS. 1 and 3. The plug-in device may then only use the portable electronic device (e.g. the cell phone 10) for assistance in presenting the information and/or for storing and transmission purposes. It is also possible to use a plug-in device without physical connections to the portable device, e.g. if the plug-in device is provided with wireless communication abilities such as e.g. Bluetooth or the like,

The earphone 20 is one sound presentation unit that can be used. As already indicated above, another type is for example a headset or headphone covering both ears.

In addition, to get an even more improved measurement quality, the portable device (e.g. the cell phone 10) may be used as a third measurement point 32″ when the portable device is in direct contact with the body of the user. The differential signal from the sensors in the plug-in device may then be averaged with the signal from the sensor in the portable device.

In general, as previously explained, it is preferred that the biometric control unit 42, arranged to perform the exemplifying method described above, is provided in the form of one or more processors with corresponding memory containing the appropriate software in the form of a program code. However, the program code can also be provided on a data carrier such as a CD ROM disc 70 as depicted in FIG. 6 or an insertable memory stick, which will perform the invention when loaded into a cell phone or some other suitable portable device having suitable processing capabilities. The program code can also be downloaded remotely from a server either outside or inside the cellular network or be downloaded via a computer like a PC to which the phone is temporarily connected.

The present invention has now been described with reference to exemplifying embodiments. However, the invention is not limited to the embodiments described herein. On the contrary, the full extent of the invention is only determined by the scope of the appended claims.

1-32. (canceled)

33. A biometric sensor system, comprising:
an information presenting unit provided with a first sound presenting unit and a second sound presenting unit to operatively present sounds to a user, wherein the information presenting unit includes a first biometric sensor unit and a second biometric sensor unit to provide signals to generate a differential measurement of one or more physical or behavioral characteristics of a user of the biometric sensor system;
a portable device provided with a sound producing unit to provide sounds to the first and second sound presenting units, the portable device including a third biometric sensor unit to obtain another measurement of the one or more physical or behavioral characteristics of the user; and
a biometric control unit to provide an average measurement of the one or more physical or behavioral characteristics of the user based on the differential measurement and the other measurement.

34. The biometric sensor system of claim 33, wherein the biometric control unit is completely or at least partly arranged in the information presenting unit.

35. (canceled)

36. The biometric sensor system of claim 33, further comprising a fastening arrangement to attach at least a part of the information presenting unit to a body of the user.

37. The biometric sensor system of claim 33, wherein each of the sound presenting units operatively attaches to an ear of the user.

38. The biometric sensor system of claim 33, wherein the first biometric sensor unit is attached to or included in the first sound presenting unit, and the second biometric sensor unit is attached to or included in the second sound presenting unit.

39. The biometric sensor system of claim 33, wherein at least one of the biometric sensor units operatively senses a heart pulse, an EEG, an EKG, or a body temperature of the user.

40. The biometric sensor system of claim 39, wherein the biometric control unit operatively presents the sensed heart pulse in the form of audible pulse beats via the information presentation unit.

41. The biometric sensor system of claim 40, wherein the biometric control unit and the sound producing unit operatively present information to the user simultaneously via the information presentation unit.

42. The biometric sensor system of claim 33, wherein the portable device is a cellular phone that operatively connects to the information presenting unit via a wired or a wireless connection.

43. The biometric sensor system of claim 33, wherein the information presenting unit is an earphone that operatively connects to the portable device via a wired or a wireless connection.

44. The biometric sensor system of claim 33, further comprising another information presentation unit in the form of a display to present visible information to the user associated with the characteristic of the user.

45-51. (canceled)

52. A method of measuring a physical or behavioral characteristic of a user using a biometric sensor system including an information presenting unit provided with first and second sound presenting units and first and second biometric sensor units, a portable device provided with a third biometric sensor unit and a sound producing unit to provide sound to the sound presenting units, and a biometric control unit, the method comprising:
measuring the characteristic using a first signal from the first biometric sensor unit and a second signal from the second biometric sensor unit to generate a differential measurement of the physical or behavioral characteristic;
performing another measurement of the physical or behavioral characteristic using a signal from the third biometric sensor unit; and
outputting, using the biometric control unit, an average measurement of the physical or behavioral characteristic based on the other measurement and the differential measurement.

53. The method of claim 52, further comprising:
controlling the measuring by the biometric control unit, wherein the biometric control unit is completely or at least partly arranged in the information presenting unit.

54. (canceled)

55. The method of claim 52, further comprising:
fastening at least a part of the information presenting until to a body part of the user.

56. The method of claim 52, further comprising:
attaching the first and second sound presenting units to ears of the user.

57. The method of claim 52, further comprising:
performing the measuring by the first biometric sensor unit that is attached to or included in the first sound presenting unit, and the second biometric sensor unit that is attached to or included in the second sound presenting unit.

58. The method of claim 52, wherein the characteristic comprises a heart pulse, an EEG, an EKG, or a body temperature of the user.

59. The method of claim 58, further comprising:
presenting the sensed pulse in the form of audible pulse beats via the information presentation unit.

60. The method of claim 59, further comprising:
presenting information from the biometric control unit and the first and second sound producing units simultaneously to the user via the information presentation unit.

61. The method of claim 52, wherein the portable device is a cellular phone that operatively connects to the information presenting unit via a wired or a wireless connection.

62. The method of claim 52, wherein the information presenting unit is an earphone that operatively connects to the portable device via a wired or a wireless connection.

63. A computer program product stored on a computer usable medium, comprising a readable program to cause a portable device to execute instructions, when said readable program is loaded in the portable device that includes an information presenting unit provided with first and second sound presenting units to present sounds to a user, a sound producing unit to provide sound to the sound presenting units, and a biometric control unit to control the measurement, the readable program comprising:
instructions to measure a characteristic of the user using at least a first biometric sensor unit and a second biometric sensor unit associated with the information presenting unit to generate a differential measurement;
instructions to perform another measurement of the characteristic using a third biometric sensor unit associated with the portable device; and
instructions to output, using the biometric control unit, an average measurement of the characteristic based on the other measurement and the differential measurement.

64. A computer program element having a program recorded thereon, where the program is to cause a portable device to execute, when the program is loaded in the portable device that includes an information presenting unit provided with first and second sound presenting units to present sound to a user, and a sound producing unit to provide sound to the sound presenting units, and a biometric control unit to control the measurement, the program comprising:
instructions to measure a characteristic of the user using a first biometric sensor unit and a second biometric sensor unit associated with the information presenting unit to generate a differential measurement;
instructions to perform another measurement of the characteristic using a third biometric sensor unit associated with the portable device; and
instructions to output using the biometric control unit, an average measurement of the characteristic based on the other measurement and the differential measurement.
